(19) United States
(12) Patent Application Publication
(10) Pub. No.: US 2026/0263281 A1
(43) Pub. Date: Sep. 10, 2026

(54) Waterproof Ear Protection Device

(71) Applicant: Alvin Hodges, Columbus, GA (US)

(72) Inventor: Alvin Hodges, Columbus, GA (US)

(21) Appl. No.: 19/321,583

(22) Filed: Sep. 8, 2025

Related U.S. Application Data
(60) Provisional application No. 63/766,419, filed on Mar. 4, 2025.

(51) Int. Cl. A61F 11/14

(52) U.S. Cl. CPC A61F 11/14

(57) ABSTRACT

The present invention is a waterproof ear protection device for preventing water ingress into the ear during bathing, swimming, or other wet activities. The device includes a dome-shaped, concave main body formed from flexible, stretchable, and water-impermeable material. An integrally formed peripheral sealing rim surrounds the inner edge of the body and is configured to conform to the user's ear, creating a secure, watertight seal without adhesive or insertion into the ear canal. The inner surface of the device is contoured to enclose anatomical features of the outer ear for improved fit and comfort. In one embodiment, an acoustic damping insert is incorporated for ambient sound attenuation. In alternative embodiments, a waterproof electronic module is embedded in the main body and includes a wireless transceiver, micro-speaker, power supply, and touch-sensitive controls, enabling wireless audio playback while maintaining full waterproof integrity.

### CROSS REFERENCE TO RELATED APPLICATION

The present application claims priority to, and the benefit of, U.S. Provisional Application No. 63/766,419 which was filed on Mar. 4, 2025, and is incorporated herein by reference in its entirety.

### FIELD OF THE INVENTION

The present invention generally relates to personal care and protective devices. More specifically, the present invention relates to a reusable, non-invasive, waterproof ear protection device configured to prevent water from entering the ear canal during activities such as bathing, swimming, or showering. The invention comprises a dome-shaped, concave main body made from a flexible, water-impermeable material such as silicone or thermoplastic elastomer, and is ergonomically contoured to conform to the external anatomy of the human ear. The device includes an integrally formed peripheral sealing rim made of compressible elastomer that creates a secure, watertight seal against the skin without requiring insertion into the ear canal. In some embodiments, the device may further include a sound-dampening insert for passive noise reduction or a sealed waterproof chamber housing a wireless transceiver, speaker, and battery for wireless audio playback. Accordingly, the present disclosure makes specific reference thereto. Nonetheless, it is to be appreciated that aspects of the present invention are also equally applicable to other like applications, devices, and methods of manufacture.

### BACKGROUND

By way of background, water inadvertently enters the ear canal of individuals during routine activities such as bathing, showering, or swimming. The presence of water in the ear can lead to a range of complications, including discomfort, temporary hearing impairment, irritation, and in more severe cases, ear infections such as otitis externa. The issue is particularly concerning for individuals with heightened ear sensitivity, those with preexisting conditions such as tympanostomy tubes, or individuals recovering from ear surgeries.

Conventional solutions, such as foam earplugs or custom-molded inserts, often require insertion into the ear canal and may cause discomfort, improper sealing, or fall out during water exposure. Conventional methods can also be invasive and unsuitable for children or individuals with sensitive ear tissue. Accordingly, individuals desire a non-invasive, externally worn, waterproof ear protection device to prevent water from entering the ear during wet activities.

Therefore, there exists a long-felt need in the art for a waterproof ear protection device that prevents water ingress during bathing, showering, swimming, and other water-related activities. There is a long-felt need for a non-invasive ear cover that can be comfortably worn by users of all ages, including individuals with ear tubes, infections, or heightened sensitivity. Furthermore, there is a long-felt need for a device that does not require insertion into the ear canal, thereby minimizing irritation and the risk of improper placement or discomfort. Additionally, there is a long-felt need for a device that conforms securely to the outer ear and stays in place without adhesives or headbands. Finally, there is a long-felt need for a reusable, watertight ear cover that can optionally provide sound attenuation and wireless audio functionality without compromising waterproof performance.

The subject matter disclosed and claimed herein, in one embodiment, comprises a waterproof ear protection device including a dome-shaped, concave main body formed of a flexible, stretchable, and water-impermeable material such as silicone, neoprene, or thermoplastic elastomer. The main body is configured to enclose and seal against the contours of the human outer ear, forming a protective barrier over the ear canal. The device includes a peripheral sealing rim integrally formed along the inner circumference of the body, comprising a compressible elastomeric band that gently engages the skin to create a watertight seal without adhesives or canal insertion. The outer surface of the device may have a textured or matte finish to improve grip and reduce slippage. The device is lightweight, comfortable, and reusable, making it suitable for daily hygiene routines as well as recreational water use.

In one embodiment, the device further includes a sound-attenuation insert integrated within the main body, composed of acoustic damping material configured to reduce ambient noise transmission. In another embodiment, the device includes an integrated waterproof electronic module comprising a wireless transceiver, micro-speaker, rechargeable power supply, and a sealed electronics chamber within the main body.

In this manner, the waterproof ear protection device of the present invention addresses long-standing limitations associated with traditional earplugs and ear covers. The device provides a hygienic, comfortable, and effective solution for keeping water out of the ear canal while enhancing user experience through optional sound insulation or wireless functionality. The device is adaptable to different ear sizes, reusable, and simple to apply and remove. The device offers a non-invasive alternative to earplugs and improves safety and comfort during every day or recreational exposure to water.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a waterproof ear protection device. The device comprises a main body having a generally concave and dome-shaped structure configured to cover the entire outer ear of a user and to form a protective barrier over the ear canal opening. The main body is made of a flexible, stretchable, and water-impermeable material. An inner surface of the main body forms a recessed cavity or chamber conforming to the anatomical shape of the human outer ear, including the helix, lobe, and antihelix. A peripheral sealing rim is integrally formed along an interior-facing circumference edge of the main body; the sealing rim comprises a compressible elastomeric band configured to exert pressure against the surrounding skin to establish a water-tight seal during use.

In another embodiment, a waterproof ear protection device with integrated sound attenuation is disclosed. The device includes a main body formed of a flexible, water-impermeable material; the main body has a concave shape defining an internal cavity or chamber for receiving the outer ear of a user. A sound-attenuation insert is embedded within the main body; the insert comprises an acoustic damping material configured to reduce transmission of ambient noise to the ear canal. The main body and insert are configured to be used during water-based activities to simultaneously protect the ear from water ingress and reduce auditory input from the external environment.

In one embodiment, a waterproof ear protection device with integrated wireless audio functionality is disclosed. The device includes a main body having a concave, dome-shaped structure adapted to cover an external human ear, a peripheral sealing rim is formed along an open edge of the main body, the sealing rim is configured to form a water-tight seal around the skin surrounding the ear, a sealed waterproof chamber is integrated within the main body, a wireless transceiver module is disposed within the chamber and operable to receive wireless audio signals from an external device, a micro-speaker is disposed within the chamber and configured to direct audio output toward the ear canal of the user, a power supply is electrically connected to the wireless transceiver and the micro-speaker, the power supply is housed within the waterproof chamber, and a touch-sensitive outer surface on the main body is operable to control playback, volume, and pairing of the wireless transceiver.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of waterproof ear protection device of the present invention in accordance with the disclosed structure;

FIG. 2 illustrates a rear perspective view of the waterproof ear protection device of the present invention in accordance with the disclosed structure;

FIG. 3 illustrates an alternative embodiment of the ear protection device an integrated electronic module designed to enable wireless audio functionality in accordance with the disclosed structure;

FIG. 4 illustrates a flow chart depicting a process of using the waterproof ear protection device of the present invention in accordance with the disclosed structure; and

FIG. 5 illustrates a user wearing the waterproof ear protection device of FIG. 1 while doing a water activity in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a waterproof ear protection device that prevents water ingress during bathing, showering, swimming, and other water-related activities. There is a long-felt need for a non-invasive ear cover that can be comfortably worn by users of all ages, including individuals with ear tubes, infections, or heightened sensitivity. Furthermore, there is a long-felt need for a device that does not require insertion into the ear canal, thereby minimizing irritation and the risk of improper placement or discomfort. Additionally, there is a long-felt need for a device that conforms securely to the outer ear and stays in place without adhesives or headbands. Finally, there is a long-felt need for a reusable, watertight ear cover that can optionally provide sound attenuation and wireless audio functionality without compromising waterproof performance.

The present invention, in one exemplary embodiment, is a waterproof ear protection device with integrated sound attenuation. The device includes a main body formed of a flexible, water-impermeable material; the main body has a concave shape defining an internal cavity or chamber for receiving the outer ear of a user. A sound-attenuation insert is embedded within the main body; the insert comprises an acoustic damping material configured to reduce transmission of ambient noise to the ear canal. The main body and insert are configured to be used during water-based activities to simultaneously protect the ear from water ingress and reduce auditory input from the external environment.

Reference will now be made in detail to the present preferred embodiments of the invention, examples of which are illustrated in the accompanying drawings. Wherever possible, the same reference numerals are used in the drawings and the description to refer to the same or like parts.

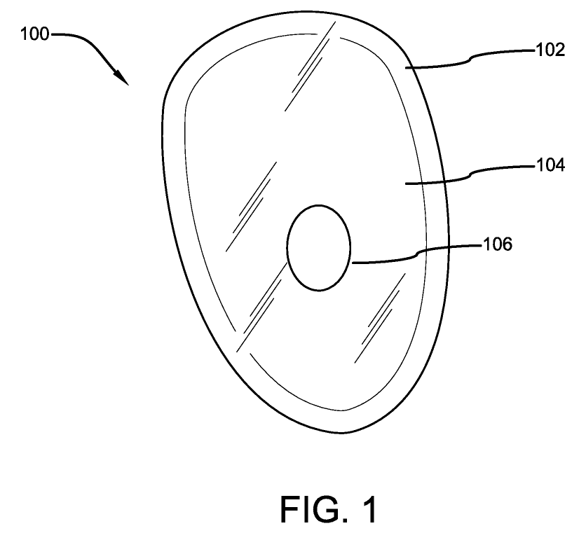

Referring initially to the drawings, FIG. 1 illustrates a front perspective view of waterproof ear protection device of the present invention in accordance with the disclosed structure. The waterproof ear protection device 100 of the present invention is designed as a specialized waterproof ear cover designed to keep water out of the ear during activities like bathing, swimming, or showering. The device 100 can be worn by individuals with ear infections, tubes, or water sensitivity to prevent water from entering the ear canal during wet activities.

More specifically, the waterproof ear protection device 100 includes a main body 102 which is designed and configured to cover the entirety of a human outer ear. The main body 102 is generally concave, dome-shaped and forms a protective barrier over the ear canal opening of a human. The main body 102 is stretchable and elastic and is preferably made from a flexible, water-impermeable material such as silicone, neoprene, latex, rubber, and thermoplastic elastomers. The outer surface 104 of the main body 102 has a textured or matte surface which helps in enhancing grip of the device 100 and reduces slippage from an ear when the device 100 is in use.

A sound-attenuation insert 106 is integrated in the main body 102 to reduce ambient sound transmission to the ear when the device 100 is worn by a user. The sound-attenuation insert 106 is preferably in the form of an acoustic damping material.

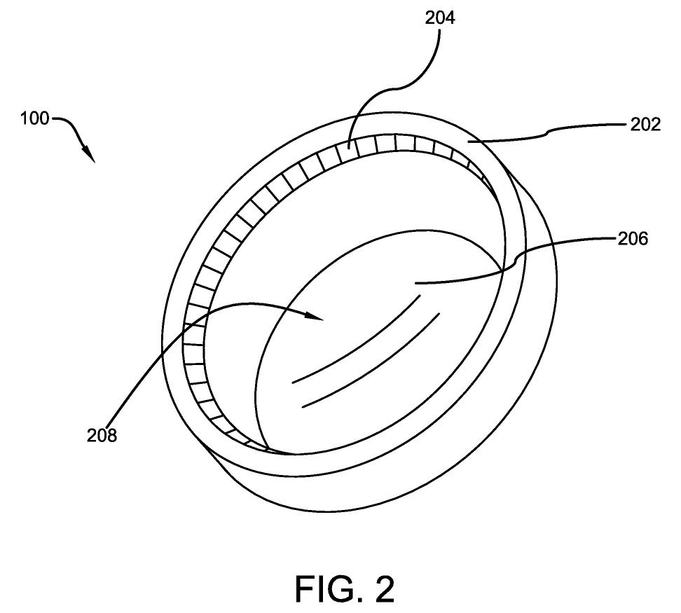

FIG. 2 illustrates a rear perspective view of the waterproof ear protection device of the present invention in accordance with the disclosed structure. The device 100 includes an interior or inner facing circumferential edge 202 which provides the concave-dome shape to the device 100. A peripheral sealing rim 204 is integrally formed along the interior or inner facing circumferential edge 202 wherein the peripheral sealing rim 204 is in the form of a compressible elastomeric band. The peripheral sealing rim 204 is dimensioned to engage with the surrounding skin around the ear in a conforming and non-irritating manner and is further, configured to exert compressive pressure against the skin of a user to establish a water-tight seal when the device is in use.

The inner surface 206 of the main body 102 is shaped and dimensioned to conform to the shape and anatomical features of the external ear of a human. The inner surface 206 forms a recessed and concave cavity or chamber 208 which encloses helix, lobe, and antihelix of a human ear. The device 100 can be worn and removed without requiring adhesive or invasive insertion into the ear canal of a human.

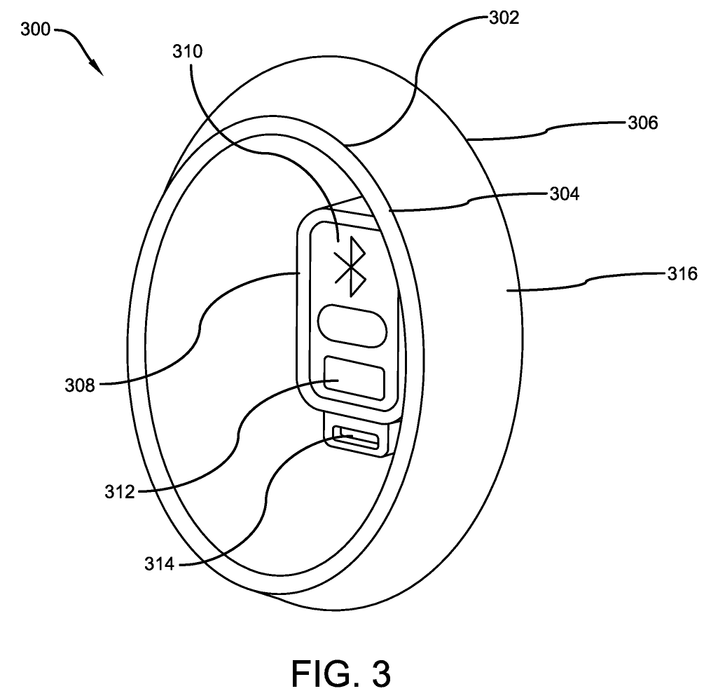

FIG. 3 illustrates an alternative embodiment of the ear protection device an integrated electronic module designed to enable wireless audio functionality in accordance with the disclosed structure. In the present embodiment, the device 300 has a dome-shaped concave body and an inner peripheral sealing ring 304 for watertight engagement when the device 100 is worn by a user. The sealing ring 304 is formed around the open periphery edge 302. The main body 306 includes a waterproof chamber 308 which is integrated into the main body 306. The waterproof chamber 308 provides a sealed space to house various electronic components.

A wireless transceiver module 310 is embedded within the chamber 308 and enables wireless communication with external devices such as smartphones or audio players. The wireless transceiver module 310 can be a Bluetooth module or can also be any conventional wireless communication technology compatible module. A micro-speaker 312 is included in the chamber 308 and is positioned such that sound playing out from the micro-speaker 312 is directed towards the ear canal.

A power supply 314 such as rechargeable lithium-ion battery or similar power source is housed within the chamber 308. The power supply 314 is electrically connected to both the wireless module 310 and speaker driver 312 and is configured to provide electric power for their operations. The outer surface 316 of the main body 306 is touch-enabled and enables a user to operate the device 300 and control playback, volume, and pairing without compromising waterproofing.

It should be noted that the device 300 of the present invention, when worn, provides both auditory protection from water and wireless audio capability. The integrated speaker 312 delivers sound through the sealed cavity or chamber directly into the user’s ear region. The waterproof chamber 308 provides watertight protection to all the internal electronics even during full submersion.

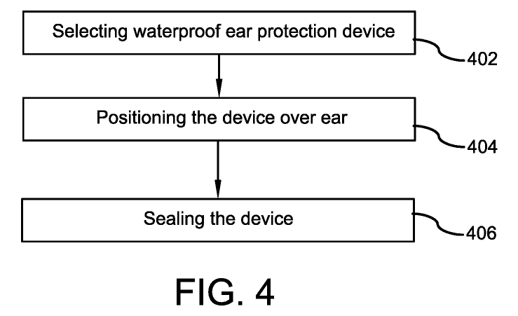

FIG. 4 illustrates a flow chart depicting a process of using the waterproof ear protection device of the present invention in accordance with the disclosed structure. Initially, based on the ear size and desired functionality, the waterproof ear protection device is selected (Step 402). Any of the devices 100, 300 can be selected by a user for use while doing a water activity. Then, the concave inner cavity or chamber of the device is positioned and centered over the external ear and the inner peripheral sealing rim gently presses against the skin around the ear (Step 404). Thereafter, optionally, the user can apply light pressure to place the device firmly, enabling the sealing rim to compress slightly and form a water-tight seal around the ear (Step 406). The device of different embodiments can be designed as reusable or disposable versions and can be easily removed after use.

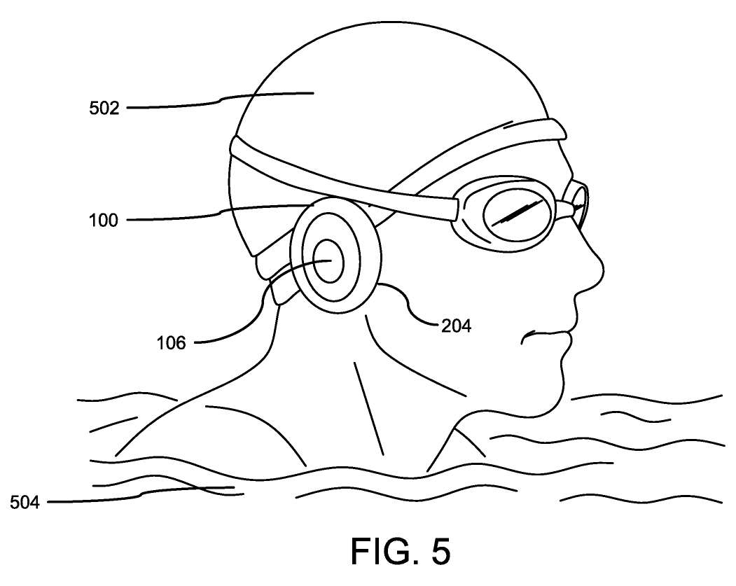

FIG. 5 illustrates a user wearing the flexible ear cover for water sealing of FIG. 1 while doing a water activity in accordance with one embodiment of the present invention. As illustrated, the user 502 wears the device 100 to enclose and seal the ear such that water 504 is prevented from entering the ear canal of the user. The device 100 does not slide or slip in use unless the sealing rim 204 is stretched and pulled by the user 502. It should be noted that the flexible ear cover for water sealing of any embodiment of the present invention can be worn by the user 502 as per preferences.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “waterproof ear protection device, “flexible ear cover for water sealing”, “ear protection device”, and “device” are interchangeable and refer to the waterproof and flexible ear protection device 100, 300 of the present invention.

Notwithstanding the foregoing, the waterproof and flexible ear protection device 100, 300 of the present invention can be of any suitable configuration as is known in the art without affecting the overall concept of the invention, provided that it accomplishes the above stated objectives. One of ordinary skill in the art will appreciate that the waterproof and flexible ear protection device 100, 300 as shown in the FIGS. are for illustrative purposes only, and that many other configurations of the waterproof and flexible ear protection device 100, 300 are well within the scope of the present disclosure. Although the dimensions of the waterproof and flexible ear protection device 100, 300 are important design parameters for user convenience, the waterproof and flexible ear protection device 100, 300 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A waterproof ear protection device comprising:
a waterproof ear cover having a main body including an inner area and an outer textured surface;
a sound-attenuation insert;
wherein said main body is concave and dome-shaped to form a protective barrier over the ear and the ear canal opening of a human;
wherein said main body having a stretchable and water-impermeable elastic material;
wherein said material is selected from the group consisting of a silicone, a neoprene, a latex, a rubber, and a thermoplastic elastomer;
wherein said sound-attenuation insert reduces ambient sound transmission to the ear when said waterproof ear cover is worn by a user;
wherein said sound-attenuation insert having an acoustic damping material;
wherein said inner area having an inner facing circumferential edge including a concave-dome shape comprising a peripheral sealing rim integrally formed along said inner facing circumferential edge;
wherein said peripheral sealing rim having a compressible elastomeric band;
wherein said peripheral sealing rim dimensioned to engage with the surrounding skin around the ear of the user;
wherein said inner area having a concave chamber for enclosing a helix, a lobe, and an antihelix of the ear of the user; and
further wherein said waterproof ear cover prevents water from entering the ear during activities selected from the group consisting of bathing, swimming, and showering.

2. The waterproof ear protection device of claim 1, wherein said compressible elastomeric band exerts compressive pressure against the skin around the ear of the user to establish a water-tight seal of said waterproof ear cover.

3. The waterproof ear protection device of claim 2, wherein said outer textured surface for enhancing grip of said waterproof ear cover.

4. The waterproof ear protection device of claim 3, wherein said sound-attenuation insert integrated with said main body.

5. The waterproof ear protection device of claim 4, wherein said inner area having a wireless transceiver module embedded within said concave chamber, and further wherein said wireless transceiver module having a Bluetooth connection for wireless communication with an external device selected from the group consisting of a smartphone and an audio player.

6. The waterproof ear protection device of claim 5, wherein said wireless transceiver module having a micro-speaker positioned to emit sound towards the ear canal.

7. The waterproof ear protection device of claim 6, wherein said wireless transceiver module having a rechargeable power supply.

8. The waterproof ear protection device of claim 7, wherein said outer textured surface is touch enabled to operate said wireless transceiver module.

9. A waterproof ear protection device comprising:
a waterproof ear cover having a main body including an inner area and an outer textured surface;
a sound-attenuation insert;
wherein said main body is concave and dome-shaped to form a protective barrier over the ear and the ear canal opening of a human;
wherein said main body having a stretchable and water-impermeable elastic material;
wherein said material is selected from the group consisting of a silicone, a neoprene, a latex, a rubber, and a thermoplastic elastomer;
wherein said sound-attenuation insert reduces ambient sound transmission to the ear when said waterproof ear cover is worn by a user;
wherein said sound-attenuation insert having an acoustic damping material;
wherein said inner area having an inner facing circumferential edge including a concave-dome shape comprising a peripheral sealing rim integrally formed along said inner facing circumferential edge;
wherein said peripheral sealing rim having a compressible elastomeric band;
wherein said peripheral sealing rim dimensioned to engage with the surrounding skin around the ear of the user;
wherein said inner area having a concave chamber for enclosing a helix, a lobe, and an antihelix of the ear of the user;
wherein said inner area having a wireless transceiver module embedded within said concave chamber;
wherein said wireless transceiver module having a Bluetooth connection for wireless communication with an external device selected from the group consisting of a smartphone and an audio player; and
further wherein said waterproof ear cover prevents water from entering the ear during activities selected from the group consisting of bathing, swimming, and showering.

10. The waterproof ear protection device of claim 9, wherein said compressible elastomeric band exerts compressive pressure against the skin around the ear of the user to establish a water-tight seal of said waterproof ear cover.

11. The waterproof ear protection device of claim 10, wherein said outer textured surface for enhancing grip of said waterproof ear cover.

12. The waterproof ear protection device of claim 11, wherein said sound-attenuation insert integrated with said main body.

13. The waterproof ear protection device of claim 12, wherein said wireless transceiver module having a micro-speaker positioned to emit sound towards the ear canal.

14. The waterproof ear protection device of claim 13, wherein said wireless transceiver module having a rechargeable power supply.

15. The waterproof ear protection device of claim 14,
wherein said outer textured surface is touch enabled to operate said wireless transceiver module.

16. A method of providing waterproof ear protection, the method comprising the steps of:
providing a waterproof ear cover having a main body including an inner area and an outer textured surface, and a sound-attenuation insert, wherein said main body is concave and dome-shaped to form a protective barrier over the ear and the ear canal opening of a human, wherein said main body having a stretchable and water-impermeable elastic material, wherein said material is selected from the group consisting of a silicone, a neoprene, a latex, a rubber, and a thermoplastic elastomer;
reducing ambient sound transmission to the ear with said sound-attenuation insert when said waterproof ear cover is worn by a user, wherein said sound-attenuation insert having an acoustic damping material, wherein said inner area having an inner facing circumferential edge including a concave-dome shape comprising a peripheral sealing rim integrally formed along said inner facing circumferential edge, wherein said peripheral sealing rim having a compressible elastomeric band;
wherein said peripheral sealing rim dimensioned to engage with the surrounding skin around the ear of the user;
wherein said inner area having a concave chamber for enclosing a helix, a lobe, and an antihelix of the ear of the user; and
pushing on said textured outer surface to move said waterproof ear cover towards the ear of the user, wherein said waterproof ear cover prevents water from entering the ear during activities selected from the group consisting of bathing, swimming, and showering.

17. The method of providing waterproof ear protection of claim 16, wherein said compressible elastomeric band exerts compressive pressure against the skin around the ear of the user to establish a water-tight seal of said waterproof ear cover.

18. The method of providing waterproof ear protection of claim 16, wherein said inner area having a wireless transceiver module embedded within said concave chamber, and further wherein said wireless transceiver module having a Bluetooth connection for wireless communication with an external device selected from the group consisting of a smartphone and an audio player.

19. The method of providing waterproof ear protection of claim 18, wherein said wireless transceiver module having a micro-speaker positioned to emit sound towards the ear canal.

20. The method of providing waterproof ear protection of claim 19, wherein said outer textured surface is touch enabled to operate said wireless transceiver module.
